(19) United States
(12) Patent Application Publication
(10) Pub. No.: US 2014/0363838 A1
(43) Pub. Date: Dec. 11, 2014

(54) MICROPERFUSION IMAGING PLATFORM

(71) Applicant: William Marsh Rice University, Houston, TX (US)

(72) Inventors: John T. MCDEVITT, Houston, TX (US); Timothy J. ABRAM, Houston, TX (US)

(21) Appl. No.: 14/299,908

(22) Filed: Jun. 9, 2014

Related U.S. Application Data
(60) Provisional application No. 61/833,750, filed on Jun. 11, 2013.

(51) Int. Cl. G01N 33/50

(52) U.S. Cl. CPC G01N 33/5005
USPC 435/29; 435/288.7

(57) ABSTRACT

This disclosure describes the design and function of a microfluidic device for performing time course assays on 2D or 3D-cell culture models designed to interface with existing robotic, high-throughput instrumentation. The system, methods and devices described herein allow robotic handling of multiplex assay cards that allow continuous fluid flow past the cells or tissues, thus allowing time course complex assays of whole cell monolayers, tissues, or 3D cell cultures.

### PRIOR RELATED APPLICATIONS

This application claims priority to 61/833,750, filed Jun. 11, 2013 and expressly incorporated by reference herein for all purposes.

### FEDERALLY SPONSORED RESEARCH STATEMENT

This invention was made with government support under Grant Number 1RC2-DE020785 awarded by the National Institutes of Health. The government has certain rights in the invention.

### FIELD OF THE DISCLOSURE

The disclosure generally relates to microfluidic platforms for performing assays on 2D or 3D-cell models over time, thus requiring perfusion of medium and or test reagents over the cells, particularly to platforms that occupy the same footprint as a standard microplate for compatibility with automation equipment.

### BACKGROUND OF THE DISCLOSURE

A patient with cancer faces two threats—the cancer itself and the cancer treatment. The adverse effects of treatment are among the highest price tags in all of medicine. The management of cancer often involves making choices among various treatment options, some of which are more toxic than others. Modern cancer therapy employs multiple aggressive treatment modalities associated with significant short and long-term morbidity. Balancing the cancer itself and the cancer treatment for a net therapeutic benefit is a judgment that requires reliable and readily interpretable information regarding both survival and toxicity.

Further complicating the process is that tumors can develop resistance to drugs. For example, a drug may be highly effective when it is first introduced to the patient, killing tumor cells and reducing the size of the tumor such that the patient goes into a remission. However, the tumor may regrow after a period of time, and this time the same drug may be less effective at killing the regrown tumor cells. This phenomenon of acquired resistance is believed to be due to a small population of drug resistant cells in the tumor that survive the initial drug treatment while the majority of the tumor is killed. These resistant cells eventually grow back to form a tumor comprising essentially only drug resistant cells.

Chemoresistant assays, based on the same principles as the chemosensitivity assay, can be performed to evaluate whether tumor growth is inhibited by a known chemotherapy drug or, more commonly, a panel of drugs. However, no current in vitro models or animal models adequately capture the complex responses of human tissues to drugs and environmental agents. Furthermore, no in vitro model captures the complex biology of tumor cell interactions with adjacent normal tissues.

Until recently, basic research testing of anti-cancer drugs was performed on cells grown on two-dimensional (2D) platforms. An emerging view holds that traditional 2D cell culture may not accurately mimic the three-dimensional (3D) environment in which cancer cells reside. Specifically, the unnatural 2D environment may provide inaccurate data regarding the predicted response of cancer cells to chemotherapeutics. Examples of differences in 2D v. 3D based assays include:
Tumor cells in 3D adopt different morphologies than on 2D.
Tumor cells cultured in 3D grow more slowly when compared to the same cells cultured on 2D platforms.
Tumor cells show increased glycolysis in 3D and often display a different gene expression profile.
Cancer cells cultured in 3D also show differences in anti-cancer drug sensitivities when compared to 2D culturing.

However, 3D tumor modeling has yet to become mainstream. Typical patient tumor samples are too small to perform multiplexed assays against combinatorial drug regimens, and the instrumentation to perform such assays is limited to large research hospitals or contract clinical research organizations.

As such, there are few tools to identify chemoresistance at the individual-patient level. As a direct result, personalized drug toxicity testing for applications such as cancer chemoresistance prediction has failed to be adopted into standard practice.

High Content Analysis (HCA) is an ideal assay for analyzing 3D models because HCA algorithms can provide measures of fluorescence intensity changes, fluorescence distribution (i.e. nuclear translocation assays), morphology, and cell movement. However, performing HCA on 3D models presents new challenges regarding image acquisition and processing before accurate regions of interest can be created.

McDevitt et al. programmable-bio-nano-chip (pBNC) platform of 61/498,761 and US20120322682, for example, has been used to perform live and fixed cellular imaging on a microfluidic scale that helps to reduce reagent consumption, and improve transport of fluorescent reagents to 3D-culture models to serve as an integrated imaging platform.

The programmable bio-nano-chip (p-BNC) system is a medical microdevice capable of delivering high performance with reduced cost associated with point of care testing. The fully integrated, total analysis system is “programmable” in the sense that the platform can be adapted to measure any combination of analytes by incorporating different biological reagents (e.g., capture and detection antibody combination).

However, cellular assays that require the sequestration of different reagents, such as drug screening assays, cannot be performed on the current bio-nano-chip system, which is optimized for a single fluidic path to react over an array of individually selected micro-reactors, in this case 3D-culture models. Furthermore, the existing cards were not designed to allow time course experiments, wherein cells are continuously (or sequentially) provided with fresh medium and/or test agents and allow the repeat taking of measurements over time.

Therefore, there is a need to incorporate fluidic dosing with a microfluidic imaging platform for performing cellular assays on 2D or 3D-culture models. Furthermore, the adoption of such a platform would be improved by designing the system in a common microplate footprint to ensure compatibility with robotic liquid handling and imaging instrumentation.

### SUMMARY OF THE DISCLOSURE

This disclosure relates to a device that can be utilized to perform cellular assays on 2D- or 3D-culture models with continuous or sequential reagent infusion while still being compatible with standardized automation equipment for sample handling and data collection. Such a device finds use in many cell-based applications, including drug discovery/drug screening, target validation, toxicity screening, wound healing assays, and evaluations of personalized treatments.

The advantages of using a microfluidic device to perform such assays include time compression resulting from non-diffusion-limited mass transport, increased mechanical stimulation of cell models, and compartmentalization of single 3D-cellular models resulting in simplified protocols for fixing and staining cells.

Another advantage of the present device is that it can replace more expensive and complicated tools currently used, which are typically relegated to either large pharma or academic centers. The standardized size of the present device will open the availability of such assays to mainstream healthcare because of its compatibility with commercially available systems presently in place and used by many healthcare providers.

Furthermore, this device has the potential to serve as a personal cancer therapy selection tool. Personalized drug toxicity testing for applications such as cancer chemo-resistance prediction has failed to be adopted into standard practice for a variety of reasons. Primarily, typical patient tumor samples are too small to perform multiplexed assays against combinatorial drug regimens, and the instrumentation to perform such assays is limited to large research hospitals or contract clinical research organizations. As such, there are few tools to identify drug-resistance at an individual-patient level. The present device will overcome many of these challenges.

The disclosure provides a device for performing microfluidic 2D or 3D-cell-culture assays using 3 components: a reusable fluidic manifold, a disposable chip with individually addressable micro-wells, and a chip holder that is compatible with standard automation equipment.

The chip holder has outer dimensions and registration features identical to those of a microtiter plate, e.g.,
Length 5.030 inches±0.010 (127.76 mm±0.25)
Width 3.370 inches±0.010
Standard height: 0.565 inch±0.010 (14.35 mm±0.25) (heights can vary)
e.g., 4×6 for a 24 well arrangement, 2×3 for a 6 well arrangement

The reusable fluidic manifold has tapped inlet and outlet holes for threaded tubing connectors and compressible o-rings at the chip interface. Both the chip holder and fluidic manifold are composed of computer numeric controlled (CNC)-milled cast PMMA, fluidic connectors and tubing, but other materials and methods can be used to make the manifold. The manifold provides a reusable component that allows external fluid sources to be easily coupled to the disposable card, allowing the easily deliver of medium and test agents to the cells. Such reagents can be delivered over time, e.g., either continuously or sequentially or combinations thereof.

The disposable chip (aka cartridge or card) is designed to encompass the micro-well array and microfluidic channels and the actual assay is performed on this disposable component. The disposable chip is exemplified herein as a 10-layer laminate device composed of alternating DSA and PET layers as well as PVDF membranes, a layer of laser-cut PMMA, and thermoformed plastic micro-wells. However, the layouts and layers used in construction can vary, as taught herein.

An initial prototype design of the disposable chip relied on in-house fabrication of thermoplastic micro-wells through a rapid vacuum thermoforming process using a precision CNC-milled aluminum mold that had a 3×4 array of positive pyramidal-shaped features micromachined into the mold. Another prototype used a transparent base material and an upper layer with etchings to form the walls of inlets and a channel or well. Any optically suitable material can be used, including glass, crystal, COC, COP, PMMA, thermoset ADC, and the like can be used.

However, other methods of making a chip with fewer layers are also possible. For example, the use of other welding, clamping or adhesive techniques can eliminate the use of DSA layers, and the micromachining or etching of layers on both sides can reduce the number of layers as well. See e.g., U.S. Ser. No. 14/258,770, filed Apr. 22, 2014 (claiming priority to 61/815,305, filed Apr. 24, 2013).

The disposable chip is assembled into separate top and bottom components, where the top component comprise the microfluidic network, venting membranes, and support layers, and the bottom components comprise the open “micro-wells” adhered to their support layers. These separate components can be sterilized individually or together, depending on assay needs. The bottom assembly can also be treated with oxygen plasma to increase the hydrophilicity of the thermoplastic micro-wells if desired, or any passivation or blocking agent can be used, as needed to prevent the assay from being hindered by the materials. Additionally, dried reagents can be encapsulated on the layers, e.g., in the fluid pathway.

By assembling the disposable chips in two primary components (upper and lower), culture models can be transferred directly to the micro-wells via automated pipetting systems. This simplifies assay initiation. Additionally, direct access to open faced wells allows larger materials such as microcarriers, scaffolds, and tissue can be added directly to the wells, thus avoiding clogging microfluidics. The separate component approach also allows various different micro-wells to be made throughout the fabrication process, such as black wells for fluorescence assays or opaque, white wells for luminescence applications, or different materials can be used for different assay types.

The card assembly mechanism itself can be reversible, such that the card can be opened, allowing the use of e.g., a multipipettor to change out reagents or allow removal of the same, e.g., fixed cells for a different type of analysis. Alternatively, the card can be closed throughout the assay

Once the upper and lower card components are assembled into the complete card, the fluidic manifold is capable of delivering multiple fluids to individually addressable microfluidic channels within the disposable chip and existing pumping equipment and connectors can be used. Current prototypes have employed 6 individually addressable channels with 3 replicate wells per channel, but next generation designs will include more channels and wells in the same overall device footprint.

The device, with its microtiter plate compatible chip holder, allows the user to take advantage of existing commercially available robotics strategies to handle the device. Robotics include instruments design to aspirate or dispense fluids, as well as instruments designed to handle plates, such as plate “grabbers”, “movers”, “shakers”, and “stackers”. Because of the availability of these instruments, there are possible variations in ways to control the fluids.

In one strategy, samples and reagents would be handled with a separate 96-well microtiter plate with wells serving as interim transfer of samples from their collection tubes. Reagents can be prepared in bulk and kept in bottles or tubes interfacing with the liquid dispenser, or kept dry on a pre-made microtiter plate format and reconstituted, then added to the preparation microplate or the modified plate with p-BNC features.

The invention includes any one or more of the following embodiments in any combinations:

A disposable labcard comprising:

| | |
|---|---|
| a) | a multilayered top compartment and a multilayered bottom compartment held in |
| | leak-proof juxtaposition and comprising a plurality of fluidic pathways; |
| b) | wherein said top compartment comprises inlets, vents and outlets, |
| c) | said bottom compartment comprises a plurality of wells, each well having an |
| | open top and a flat transparent bottom; |
| d) | wherein each fluidic pathway includes at least one inlet, at least one vent, at |
| | least one well and at least one outlet. |

A disposable labcard wherein said fluidic pathway comprised a plurality of inlets each
connected to a vent, and said plurality of inlets merge to connect to a single well.
A disposable labcard wherein each said fluidic pathway comprises at least two wells in
series allowing each assay to be performed in duplicate.
A disposable labcard wherein said fluidic pathway comprises at least two wells in series
allowing each assay to be performed in duplicate.
A disposable labcard wherein each said fluidic pathway comprises at least three wells in
series allowing each assay to be performed in triplicate.
A disposable labcard wherein each said fluid pathway comprises at least three wells in
series allowing each assay to be performed in triplicate.
A disposable labcard wherein said multilayered top compartment and a multilayered
bottom compartment each comprise polymeric layers.
A disposable labcard wherein said polymeric layers are selected from a group
consisting of DSA (double sided adhesives), PET (polyethylene terephthalate), PMMA
(poly(methyl methacrylate)), cyclic olefin co-polymer (COC) and PVDF (polyvinylidene
difluoride).
A disposable labcard wherein layers are held in fluid tight juxtaposition by adhesives,
solvent welding, and other chemical means. Mechanical means can also be used, such
as separate clamps, rivets, screws and other mechanical means, or snap fit and other
mechanical connectors can be integral to one or more layers. Thermal welding and
other irradiative means could also be used, such as RF welding, laser welding, spot
welding, and the like. Combinations of all of these are possible, and especially
preferred are chemical means for affixing the layers within the separate upper and lower
subcomponents, and a mechanical means for affixing the upper and lower components
together.
A disposable labcard wherein said polymeric layers are alternating layers of DSA and
PET.
A disposable labcard wherein each vent comprises a gas pathway to an upper surface
of said disposable card above an inlet pathway, said gas pathway having a porous
membrane thereacross allowing gas passage therethrough.
A disposable labcard wherein each well comprises a plurality of microcarrier beads or
other scaffold material for growing cells on surfaces thereof.
A disposable labcard wherein wells are made by thermoforming a transparent polymer
to form wells and well walls.
A disposable labcard wherein wells are made by affixing a transparent base layer that
forms well bottoms to a layer having holes therein, said holes forming or delineating well
walls, the height of the layer (or layers) determining the depth of the well.
A microassay platform comprising:

| | |
|---|---|
| a) | the mutilayered disposable labcard as herein described; |
| b) | a card holder having a pocket for said disposable labcard and having a footprint |
| | (length × width) of a standard microtiter plate; |
| c) | a reusable fluidic manifold located above said disposable labcard and in fluid |
| | connection with said disposable card and having leakproof fluidic connections |
| | with one or more outside fluid sources or connectors for same; and |
| d) | holding means for holding said labcard holder, said disposable card, and said |
| | reusable fluidic manifold together. |

A platform for performing assays, said platform comprising:

| a) | a reusable fluidic manifold; | |
|---|---|---|
| b) | a disposable card having a plurality of fluidic pathways; | |
| | i) | wherein said disposable labcard comprises a multilayered top compartment |
| | | and a multilayered bottom compartment; |
| | ii) | wherein said top compartment comprises inlets, vents and outlets; |
| | iii) | wherein said bottom compartment comprises wells, each well having a flat |
| | | transparent bottom; |
| | iv) | wherein each fluidic pathway includes at least one inlet and vent connected |
| | | to at least one well connected to at least one outlet; |
| | v) | wherein said fluidic manifold provides a plurality of connectors, each |
| | | allowing fluid flow between an external fluid source and one of said plurality |
| | | of fluidic pathways; |

| | |
|---|---|
| c) | a card holder having a footprint of a standard microtiter plate and a pocket |
| | therein side to receive said disposable labcard; |
| d) | a means for holding said reusable fluidic manifold, said disposable labcard and |
| | said card holder together in leak proof juxtaposition. |

A platform wherein holding means are screws threaded through one or more
components, a rivet passed through one or more components, a clamp encompassing
all components, snap fit connectors integral to one or more component, and other
mechanical means tightly pressing the components together.
A platform wherein each fluidic pathway includes a plurality of inlets connected to said
at least one well.
A platform wherein each fluidic pathway includes an inlet connected to a plurality of
wells in series.
A platform wherein each fluidic pathway includes a plurality of inlets connected to a first
well, and said first well connected to at least one second well.
A method of performing assays on cell cultures, comprising:

| | |
|---|---|
| a) | providing a platform as herein described, |
| b) | inoculating said wells with a population of cells; |
| c) | continuously perfusing a cell medium through said fluidic pathways and over |
| | said cells; and |
| d) | measuring one or more parameters of said cells over time. |

A method wherein a test agent is added to said cell medium and measuring said one or
more parameters of said cells before and after said test agent is added.
A method wherein said more or more parameters is selected from cell number, viable
cell number, nuclear size, dead cell number, cell area, and nuclear-to-cytoplasm area.
A method which is an assay such as toxicity screening, sensitivity screening, drug
testing, target validation, wound healing assays, cell culture optimization assays,
antibiotic sensitivity testing, hormone testing, cell mobility assays, and the like.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| ADC | allyl diglycol carbonate |
| CNC | Computer numeric controlled |
| COC | Cyclic olefin co-polymer |
| COP | Cyclo-Olefin Polymer |
| DSA | Double sided adhesive |
| PET | Polyethylene terephthalate |
| PMMA | Polymethyl-methacrylate |
| PS | Polystyrene |
| PVDF | Polyvinylidene difluoride |
| ROI | Region of interest |

### BRIEF DESCRIPTION OF DRAWINGS

FIGS. 1A-D illustrate different aspects of a micro-well assembly of this disclosure.

FIG. 1A shows an exploded view of the labcard, where the dotted lines illustrate the fluid path through the various layers. An upper sub-compartment housing the inlets, outlets, and vents can be assembled as an upper subunit of the disposable card. If so, a lower sub-compartment housing the wells is also assembled. The wells can thus be filled, e.g., with microcarriers or other cell scaffold materials, the two sub-compartments brought to together and e.g., clamped for use. If direct access to the wells is not needed, the entirety of the layers can be assembled at the same time.

FIG. 1B shows a top view of the disposable labcard, with a dotted line representing the position of a cross-section view in FIGS. 1C and 1D.

FIG. 1C is a cross-sectional view of the three-dimensional attributes of the micro-well assembly shown in FIG. 1B.

FIG. 1D is another cross-sectional view illustrating the fluid path through the assembled device from inlet (112) to inlet channel (118) to a micro-well (124), to the bottom well-to-well channel (123), up the ascending fluid port (122), to the to well-to-well channel (119), to the two downstream micro-wells, to the outlet (12), and up through the outlet (113). Here, 3 wells are in the same fluid path, allowing each assay to be performed in triplicate. Further, the card design allows continuous perfusion of the wells, thus allowing for the study of live cells or tissues over time in a pNBC platform using standard microtiter plate handling equipment.

FIG. 2A-E depicts the assay-card being used with a manifold. FIG. 2A shows the subsequent assembly with the same disposable labcard illustrated in FIG. 1 in combination with the dosing manifold (201) and the card-holder (203).

FIG. 2B shows a top-down view of a possible embodiment of the dosing manifold displaying the location of the cross-sectional view in FIG. 2C.

FIG. 2C is a cross-sectional view of the dosing manifold where the machined fluid ports that relay the fluid from the tubing adapter down to the inlet of the disposable assay card, and from the outlet of the card back to the outlet tubing in the manifold.

FIG. 2D is a top-down view of the assay-card holder that shows the location of the cross-sectional view in FIG. 2E.

FIG. 2E is an isometric cross-sectional view of the assay-card holder, which shows how the card pocket is designed to securely adapt the disposable assay-card.

FIG. 3 shows another possible embodiment of the disposable assay-card wherein three fluidic inlets connect with a single elongated chamber, allowing different fluids to perfuse the cell chamber, either at the same time or sequentially. As with the embodiment in FIG. 1A-D, the lower layers (207, 208) can be assembled as a sub-compartment if direct access to the wells with large components, such as scaffold, microcarriers, or e.g., tissue slices is needed. In this embodiment, the wells are made with a transparent base and second layer forming the walls of the wells, wherein in FIG. 1A-D the wells were made by thermoforming a transparent polymer into the correct shape for wells. Etching or drilling are other means of making wells with an open face and transparent base.

FIG. 4A-C depict the alternative disposable assay-card being used with the manifold and card-holder. FIG. 4A is an exploded view illustrating the fully assembled assay unit with the gravity-fed media-dosing manifold, the disposable assay-card, and the card holder.

FIG. 4B is a top view of the gravity-fed media-dispensing manifold and shows where the cross-sectional view for FIG. 4C originates.

FIG. 4C is an isometric cross-sectional view showing the parabolic media wells and how they connect to the machined O-ring pocket.

FIG. 5 shows the imaging sequence of the microcarrier assay, wherein A) is the Raw image stack, B) is the Maximum Intensity Projection (MIP) of deconvoluted raw stack, C) is the bandpass filtered image with background subtracted, D) is the identified objects (nuclei) by Cell Profiler, E) is the superimposed cell nuclei outlines over original image from (C).

FIG. 6 shows the cell growth on microcarriers in the pNBC card of FIG. 1.

FIG. 7 shows comparative dose-response curves for MDA-MB-468 cells to doxorubicin in both a monolayer format using a conventional fluorescence plate reader and a microcarrier format using the pBNC device to perform live/dead cell assays.

FIG. 8 shows cell viability curve based on counted numbers of identified live and dead cells from a calcein-AM/EthD-1 assay. The dashed line is the average bBNC dose curve from FIG. 7 for comparison.

FIG. 9 shows the total cell counts for microcarrier and monolayer assays based on identified nuclear staining.

FIG. 10 shows changes in nuclear area with increasing doxorubicin concentration.

FIG. 11 shows the measurement of nuclear compactness computed from identified nuclear regions of interest (ROI) as a measure of the variance in the radial distance of an object's pixels from its centroid divided by its area.

FIG. 12 shows DAPI intensity distribution among nuclei identified in monolayer (A) and microcarrier (B) platforms across a range of doxorubicin concentrations.

FIGS. 13A-B show controlled sequestered reagent delivery via laminar flow.

FIG. 13A are time-lapse images demonstrating the ability to control the position of two separate fluids (blue and yellow inks) in order to sequester a reagent to a particular channel width. The six panels represent different flow rates for the two inks, from predominantly dark ink flowing in the first panel, to an even flow rate (see arrow), to a predominantly light ink flowing is the last panel. FIG. 13B is a linear regression of channel distance as a function of the flow rate ratio between the two independent fluids (attached to independent pumping sources). Data was collected in triplicate and error bars represent one standard deviation above and below the mean for each flow rate ratio.

FIG. 14 shows the comparison between chip and static doxorubicin uptake in the wells of the labcard herein described.

FIG. 15 shows the difference between chip and plate doxorubicin uptake with different Z depths.

### DETAILED DESCRIPTION

The disclosure provides a novel device for performing assays while allowing continuous perfusion of media, drugs and other reagents past the cells. Therefore, cell response over time can be assayed in a variety of formats, such as time course experiments of traditional 2D cultures, pseudo-3D culture, e.g., using scaffolds such as microcarriers, tissues, or the device can be used for true 3D cultures of e.g., tissue slices or magnetic cells cultured in a magnetic field, such as described by Nano3D Biosciences, Inc.

In addition, the device is a microfluidic platform with the same footprint as a standard microplate that is commonly used with commercially available robotics. Thus, the system is compatible with existing robotics and fluidics.

We provide a 3-component system for performing microfluidic 3D-cell-culture assays through 1) a fluidic manifold, 2) a disposable chip with individually addressable micro-wells that can be composed of two sub-components if desired, and 3) a chip holder that is compatible with standard automation equipment.

Further the system is modular, allowing for various components to be redesigned for different uses. Thus, different cards can be provided for different assay types, and the cards could still fit with the same card holder and/or fluid manifold. Additionally, there can be more than one fluid manifold, depending on assay needs. Therefore, this application discloses a variety of disposable chip designs to perform various microfluidic-based assays.

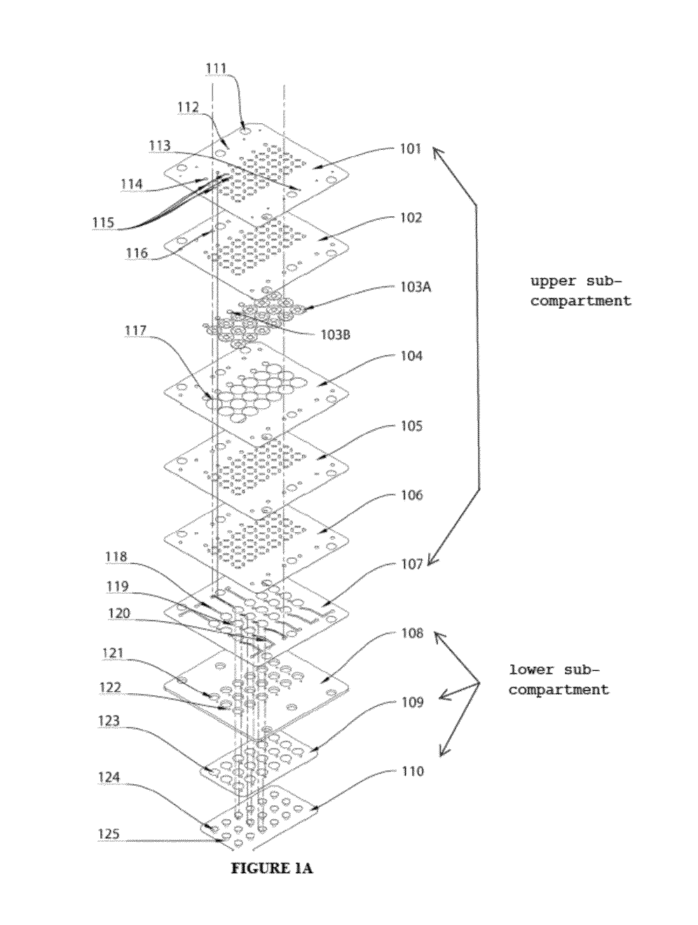

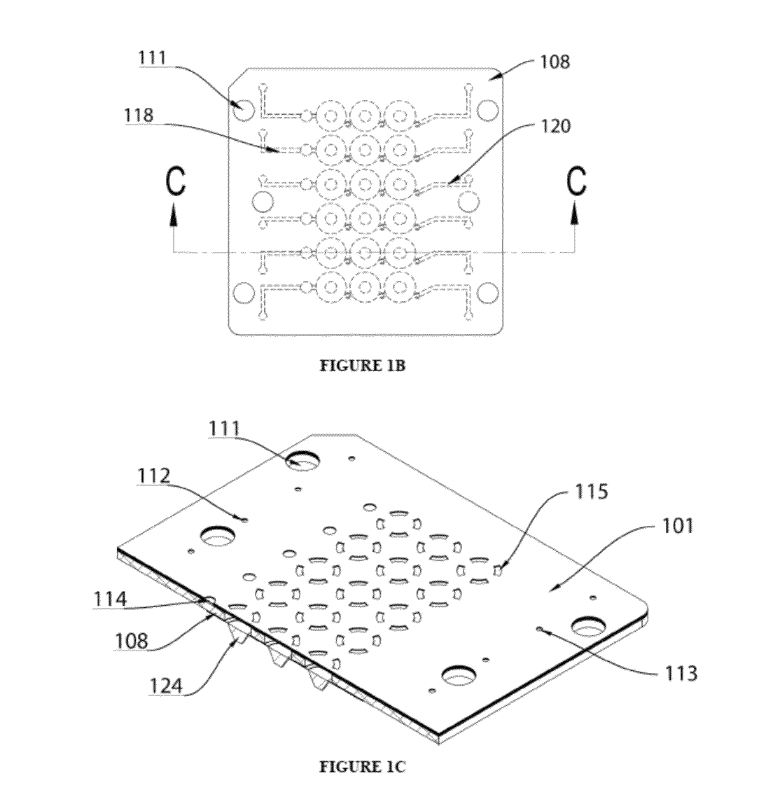

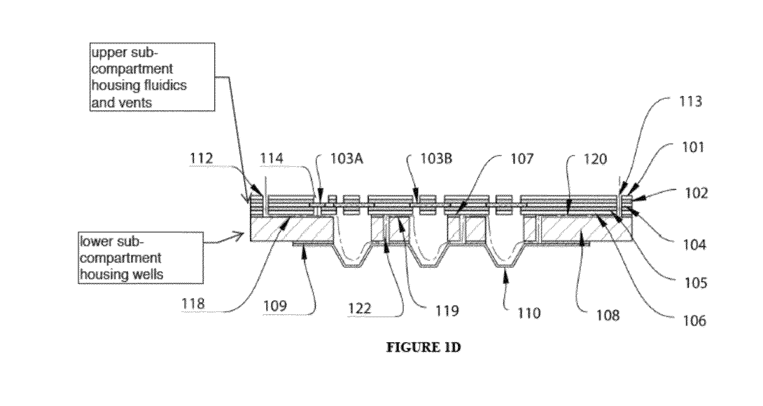

FIG. 1A is an exploded view of one perfusion assay card, wherein three wells are in the same fluid pathway, allowing each assay to be performed in triplicate using the same fluids. The dotted line illustrates the fluid path through the many layers from the inlet (112) to the inlet channels (118), to the individual wells (124) where each inlet channel connects with three separate wells, to the outlet channel (120), and up through the outlet (113). In this figure, only one fluid path has been depicted with the dotted line, but the current embodiment allows for six separate inlets each containing three separate wells (thus, two inlets per well).

This disposable chip is composed of 10 different layers and sealed by altering layers of double-sided adhesive (DSA) and PET. The layers can be assembled all at once, or if substantial well access is needed, e.g., to load the wells with tissue slices, the card is assembled into two sub-compartments—a lower sub-compartment containing open faced wells, and an upper sub-compartment containing fluidics and vents. The two sub-compartments can be combined into the final card once the wells are loaded.

When fully assembled with the fluidic manifold, fluids enter the disposable chip through the inlet holes (112), pass through and reach the microfluidic channels (118), and begin to fill the micro-wells (124). Before reaching the micro-well interface, residual air is purged from the tubing lines and inlet channels through a porous PVDF vent membrane (103b) that is supported by a top DSA layer (102) and a bottom DSA layer (105) and a bottom membrane PET support layer (106). A similar venting scheme is included for each individual well, where the current embodiment consists of donut-shaped well vent (103A) of the same venting material over each well (124).

The bottom of wells (125) is typically a flat, transparent plastic, suitable for live cell or tissue use, and we have made this layer (110) by thermoforming using a mold. This flat transparent base allows an inverted scope to be used for the assays, although the platform is also compatible with top down microscopy.

In order to support the membranes, small apertures (115) are present above and below the membranes, which allow for gas exchange between the contents of the micro-wells and the surrounding environment. After the fluid fills the first column of wells, it exits through a microfluidic outlet channel (120) on the bottom of the DSA layer (107).

The fluid then flows up through the layers until it enters the same layer (107) as the inlet channels (118) before flowing into the subsequent micro-well. The fluid is thus driven from the top of the well to the bottom of the well to allow mass transport to take place throughout the entirety of the micro-well. The chimneys that connect the outlet channels to the inlet channels serve as a passive hydrostatic valve while cellular materials are transferred to the micro-wells when the chips are open. After filling the last micro-well, fluids exit through the outlet channels (120), the outlet holes (113), and back through the fluidic manifold outlets.

The three components of the microfluidic assay platform (described below) are connected through a series of holes (111) that allow a thumb-tightened screw to pass through the top manifold and disposable chip and tighten against the chip holder, whose hole is threaded to match the screw.

FIG. 1B shows a top view of the disposable labcard, where a dotted line C-C represent the position of the cross-sectional view in FIGS. 1C and 1D.

FIG. 1C shows a cut-off view of the assay-card of this disclosure. The cross-section shows the three-dimensional relationship between the inlet holes (112), the inlet channel vent hole 114, the micro-well cavities (121), the micro-well (124) and the outlet holes (113). FIG. 1C also shows the fastener holes (111) that will later be described. The micro-wells are composed of layer (110) making thermoformed plastic wells (124) on the very bottom attached to a thin slab of PMMA (108) with corresponding well cavities (121) outlines, and assembled using a layer of DSA with the same outlines (109).

FIG. 1D shows a cross-sectional view of the fully assembled assay-card wherein upper and lower sub-compartments are clearly visible. The dotted line again indicates the fluid path from the inlet hole (112) through the inlet channel (118) into a micro-well (110), back to ascending fluid port (122), pass through top well-to-well channel (119), into the subsequent micro-well (110), and against through the corresponding ascending fluid port (122) and top well-to-well channel and then into the last of the three micro-well (110), before leaving through the outlet channel (120) and finally exit through the outlet hole (113). The inlet channel 118 runs vertically through the top cover (101), the top membrane support layer (102), and membrane spacer 104, and two bottom membrane support layers (105, 106) before horizontally connecting to the micro-well (110). Layer (109) is the layer of DSA that attaches the microwells (110) to the acrylic layer (108).

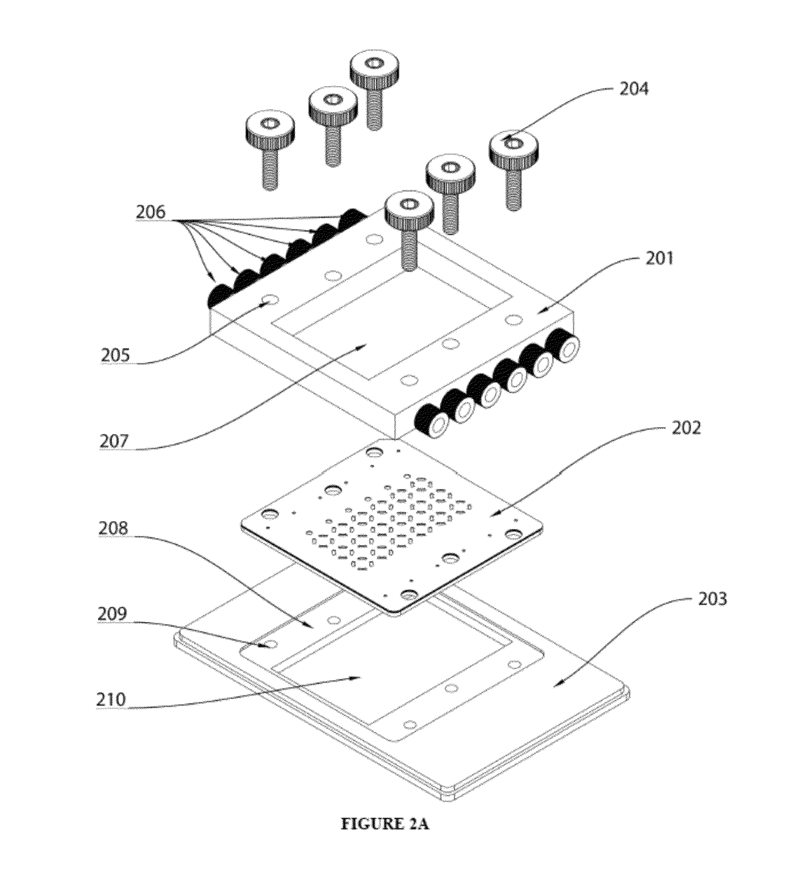

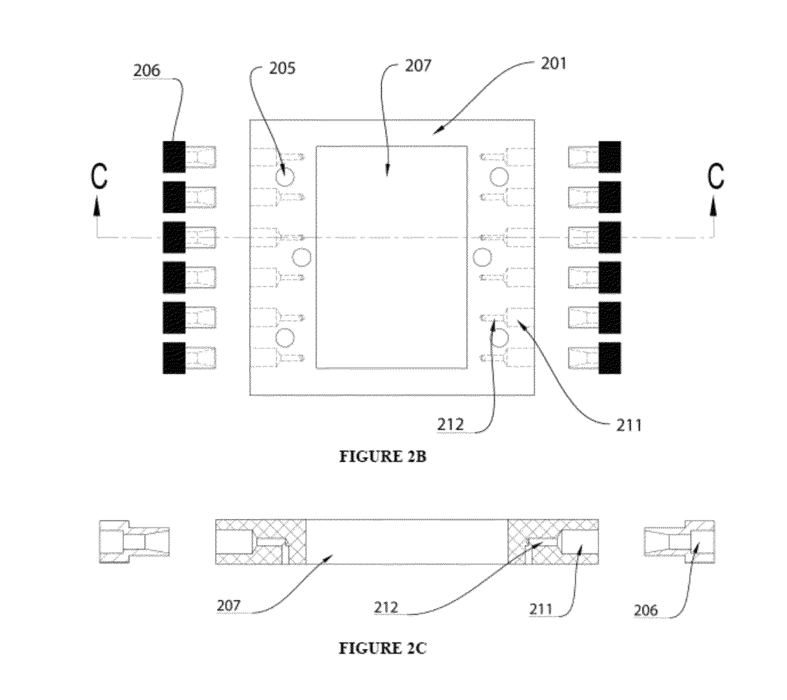

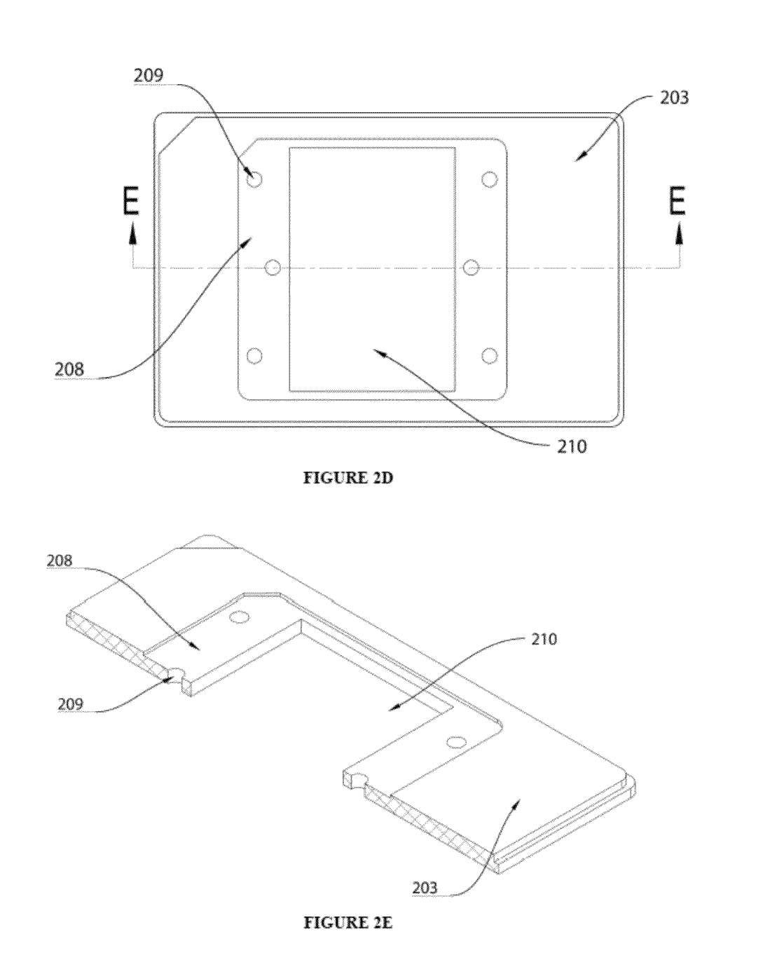

FIG. 2A shows the subsequent assembly with the same disposable card illustrated in FIG. 1 in combination with the dosing manifold (201) and the card-holder (203). The nature of this assembly method allows various manifold designs and disposable card designs to be swapped out during different stages of testing to achieve unique functions in a modular strategy. The dosing manifold 201 is made of machined acrylic and has multiple threaded tubing adaptors (206) at both ends of the manifold. An optical viewing window (207) is provided for an upright microscope, but, even if not used, the window saves material and reduces weight.

Fastener holes (205) are provided on the manifold and threaded holes (209) are provided on the bottom card holder (203) so that thumb-screw fasteners (204) can be used to securely fasten the manifold (201), the disposable labcard (202) and the bottom card holder (203) together. However, other clamping or attaching means are also possible. A pocket (208) is provided on the card holder (203) to fit the assay card (202).

FIG. 2B is a top-down view of a possible embodiment of the dosing manifold as shown in FIG. 2A. The dotted line C-C is the location of the cross-section view in FIG. 2C. As shown in FIG. 2B, fluid is propagated from the fluidic connectors to the fluid ports (211) of the disposable chip through small channels drilled into the sides of the manifold (201).

FIG. 2C is a cross-sectional view of the assembly as shown in FIG. 2A. The assembly has the threaded fluid adapter port (211) to receive the threaded tubing adapter (206), and fluid manifold port (212) is in fluid communication with the threaded fluid adapter port (211) such that the sample fluid can be introduced from the tubing adapter (206) through the fluid manifold port (212) into the assay-card.

FIG. 2D is a top-down view of the assay-card holder, wherein the location of the cross-sectional view as shown in FIG. 2E is indicated by the dotted line E-E. The card holder is designed to occupy the same footprint as an industry-standard microtiter plate in order to make the assembly compatible with automated imaging and dispensing equipment.

FIG. 2E is an isometric cross-sectional view of the assay-card holder. The figure demonstrates how the card pocket is designed to securely adapt the disposable assay-card. The threaded holes (209) accept the fasteners that pass through the dosing manifold and the disposable card to create a leak-free system. Other leak proof attachment means mechanisms could be incorporated, such as quick-release clamps, embedded neodymium magnets, vacuum suction, or small push-lock fittings, among others.

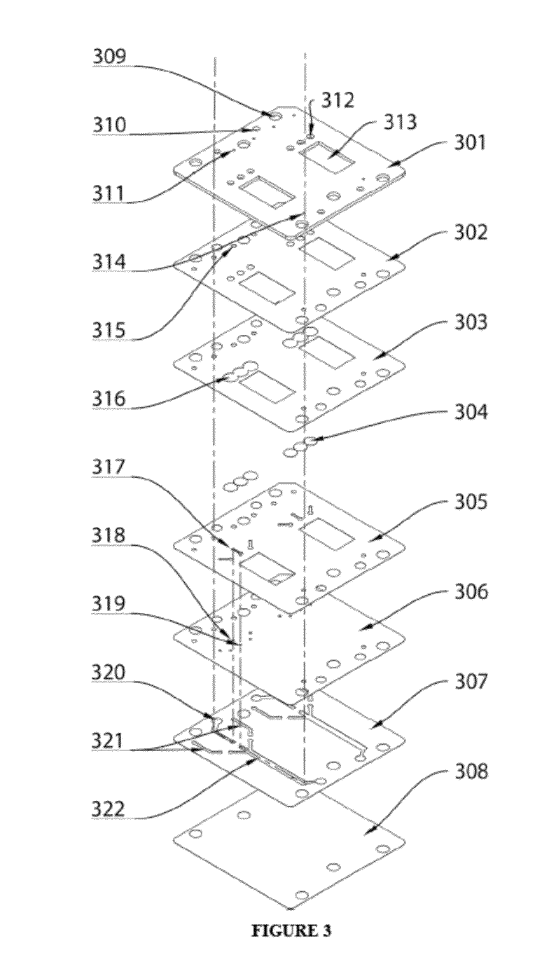

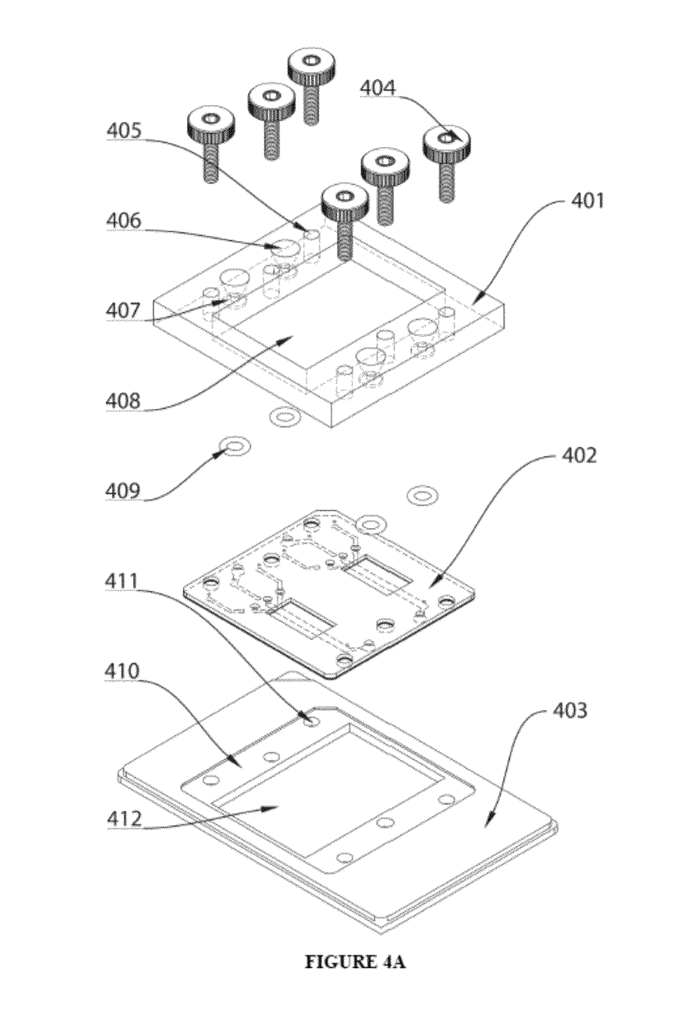

FIG. 3 shows another possible embodiment of a disposable assay-card. The card illustrated here is intended to be used for monolayer cell assays, where a flat, optical polymer layer (308) replaces the thermoformed micro-wells from the card depicted in FIG. 1. Additionally, a membrane vent channel (317) is added as a potential bubble trap, where the fluid is forced up into contact with the vent membrane before passing back down through the remaining fluidic system. This illustration depicts a card with two replicates of a single cell culture channel with 3 inlets and one outlet.

In addition to the six (6) total inlets and two outlets, there are also fluid ports that interface with a gravity-fed media dispensing manifold. The card is designed to be compatible with this manifold as well as the dosing manifold from FIG. 2B. Since compressible O-rings interface with the fluid ports, these act as a closed valve for the fluid ports that are not in use.

Beneath the top rigid support layer (301) is a top membrane support layer (302), followed by a PET vent membrane spacer (303). Between the vent membrane spacer (303) and a top venting channels (305) are several venting membranes (304) similar to that shown in FIG. 1A. Underneath the top venting channels (305) is a cell chamber cover (306) made e.g., of COC, followed by a main fluidic branching channel layer (307), and finally the cell-culture surface that is made of COC, PS or similar compatible materials.

Fastener holes (309) are similarly provided for securely fastening the layers. Gravity-fed media dosing manifold holes (310) are provided as media inlet. Fluid inlet holes (311) are provided for introducing fluid as shown in the dosing manifold shown in FIG. 2B. Air vent holes (312) allows venting, and the optical window (313) is provided for upright microscope viewing, although the cultured cells can also be viewed through the transparent lower layer (308) with an inverted scope.

Again the dotted lines indicate the fluid path in this alternative embodiment. The fluid first flow through the gravity-fed media dosing manifold (310) located on the top rigid support layer (301), down through the top membrane support layer (302), the vent membrane spacer (303), the cell membrane cover (306), and reaches the media inlet channel (320). The fluid flows across the inlet channel (320) then flows up through ascending vent holes (318) and reaches the top venting channel (317). After passing across the top venting channel (317), the fluid flows down through the descending vent hole (319) and finally reaches the cell-culture channel (322). The fluid then flows across the entire cell-culture channel (322) before existing the manifold through fluid outlet holes (314). This flow path allows for efficient and thorough reaction between the sample fluid and the reagents or probes in the cell-culture channel (322) so that a fast and accurate reading can be achieved. Such an embodiment would be useful for various time course 2D assays. For example, we have used this platform for toxicity testing, wound healing, and fixed-cell assays.

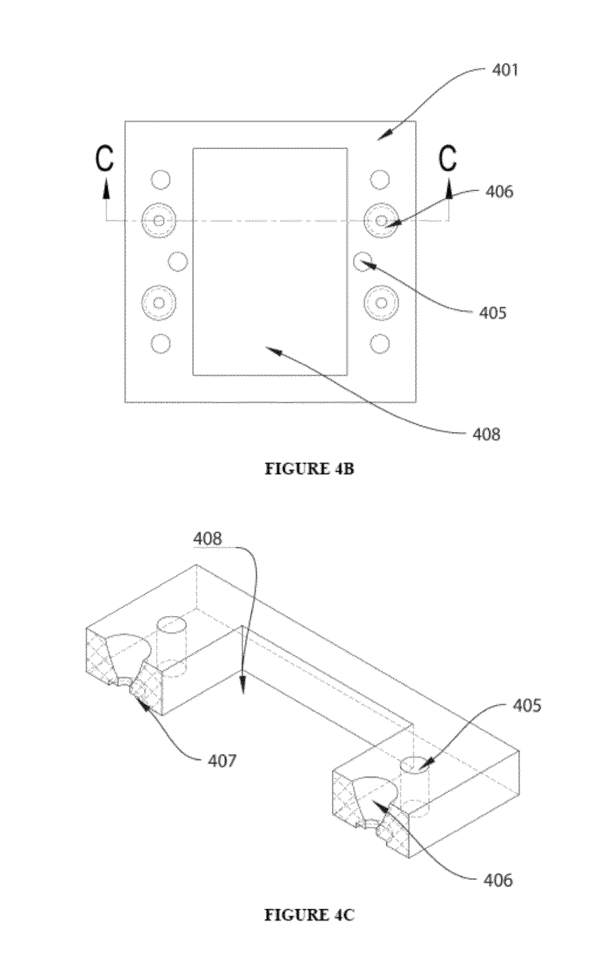

FIG. 4A is an exploded view illustrating the fully assembled assay unit with the gravity-fed media-dosing manifold (401), the disposable assay card (402) of FIG. 3 that is used for monolayer cell culture, and the card holder (403). A pocket for the disposable card (410) is provided on the card holder (403) to receive the disposable card (402). Small pockets are milled into the bottom of the fluidic manifold where the fluid inlets and outlets meet the fluidic ports on the disposable chip to accommodate the compressible O-rings (409) without the need for any adhesive.

Six thumb-screw fasteners (404) are used to secure the three layers together through corresponding fastener holes (405) on the manifold (401) and the threaded holes (411) on the card holder (403), but other attachment means could be used. An optical window (408) is provided on the manifold (401) for upright microscopes, and another optical window (412) is provided on the card holder (403) for inverted microscopes. Four parabolic media wells (406) are provided for gravity feeding media to the cells. Four O-ring pockets (407) are provided to accommodate the compressible O-rings (409).

FIG. 4B shows the top view of the assembly in FIG. 4A, and the dotted line C-C indicates the cross-section shown in FIG. 4C.

FIG. 4C is a cross-sectional view that illustrates the parabolic media wells and how they connect to the machined O-ring pockets. Fluid that fills these wells (406) is slowly dispensed over time into the media inlets on the disposable assay card, allowing for pumpless cell-culture. The modular nature of these devices allows a user to swap out the gravity-fed media-dispensing manifold for a manifold with tubing adapters for active pumping of reagents throughout the system, while maintaining the same footprint.

Materials and Methods

In order to test our devices, a number of assays were performed, and in many instances compared against a similar assay in a 96 well format.

The goal in our imaging experiments was not to perform the dosing of the drugs but to create an imaging endpoint as a surrogate for cell viability. Thus, the imaging tests used an adapted prior p-BNC platform, wherein microcarrier beads replaced the agarose beads in the bead holder array, and the cards had bottom draining outlets. The microcarriers were treated with drug in conventional culture plates and then transferred to the bead holder before administering the viability stains for the assay.

Multiple copies of a 3×4 array of positive pyramidal-shaped features were micromachined using a precision computer numeric controlled (CNC) router (HAAS) into an aluminum block.

Micro-containers were then created by replica molding using a UV-curable epoxy (NOA81, NORLAND OPTICAL™) poured over the positive aluminum mold, sandwiched by a slab of acrylic (MCMASTER™) and curing under a UV light source for 20 minutes. The resulting inverted pyramidal-pit shaped, through-hole micro-containers had dimensions of 240 μm×240 μm for the top opening and 70 μm×70 μm for the bottom opening of the wells. This process was repeated on the same aluminum master to yield over 100 micro-container arrays. The microcarriers were 200 μm in diameter and served to hold the cells in place, yet still allowing fluid passage, drainage and imaging.

Human breast cancer cells MDA-MB-468 were grown in Dulbecco's Modified Eagle Medium (DMEM) supplemented with 10% fetal bovine serum and 100 U/mL penicillin and 0.1 mg/mL streptomycin (SIGMA®). MDA-MB-468, was chosen for these experiments due to its availability and its excellent attachment and growth dynamics on the Cytodex I microcarriers. Cells were grown in 75 cm² tissue culture flasks (CORNING®) and passaged or frozen upon reaching 80% confluency. Cultures were maintained in a humidified incubator at 37° C. in 5% CO₂/95% air. Media was replenished every 48-72 hours. Frozen aliquots of cells from passages 14 and 15 were used for all experiments with the same media formulation.

Cytodex I microcarriers (GE®) were prepared according to the manufacturer's recommendations. Dry microcarriers were hydrated with PBS in a 250 mL siliconized glass bottle (CORNING®) with PBS (100 mL/g Cytodex) overnight at room temperature. Supernatant was discarded and replaced with fresh PBS. The hydrated microcarriers were sterilized by autoclaving for 20 minutes at 121° C. After microcarriers had settled, the supernatant was discarded and microcarriers were washed with warm culture media. After media was removed, microcarriers were re-suspended in fresh warm media (100 mL/g Cytodex).

Microcarrier culture is traditionally performed in suspension, in order to maximize the amount of surface available for cell attachment and growth. The ability of microcarriers to provide a high culture area/volume ratio is not limited to suspension culture, as a high yield of cells can be obtained from microcarrier cultures in traditional monolayer vessels. Because small numbers of microcarriers were utilized at a time, static culturing conditions were used to decrease the volume requirements and amount of waste for each experiment.

Scale-up of microcarrier culture is simple to perform and can lead to high yields if so desired. In a static environment, 5 mg/mL hydrated microcarriers were inoculated with 6E4-2.5E5 cells/mL, based on provided protocol recommendations of a 1-5 mg/mL Cytodex density inoculated with 5E4-2E5 cells/mL. Microcarrier cultures were incubated at 37° C. and 5% CO₂ and periodically agitated on a plate shaker (VWR) to prevent microcarriers from attaching to the tissue culture well bottoms. Media was replenished every 48-72 hours.

Microcarriers cultured with MDA-MB-468 cells were harvested after 5-6 days of static culture with regular media exchanges every 48 hours. Doxorubicin solutions were prepared in fresh, warm media in volumes of 1 mL and added to aliquots of about 200 μL microcarriers and statically incubated for 24 hours at 37° C. Each aliquot was transferred to a sterile 2 ml micro-centrifuge tube where the microcarriers were allowed to settle. After supernatant was removed, microcarriers were rinsed in warm PBS, and incubated in fresh media at 37° C. until assayed. Negative control microcarriers were prepared by replacing this media with 70% methanol and incubating at room temperature for 30 minutes, and then suspended in PBS.

For each assay performed in triplicate (on a prior card embodiment with outlets at the bottom of the card), 4 negative control microcarriers were placed in the 4 outer corners of a 3×4 array of micro-containers through careful manipulation with precision forceps. The remaining 8 locations were loaded with microcarriers of the doxorubicin dosage to be tested. After loading of the microcarriers, the flow cell was assembled, resulting in a leak-free system to direct fluid flow (e.g., fresh media and/or test agents) across the tops of the microcarriers, into a reservoir below the micro-containers, and out a bottom outlet.

A live-dead fluorescent viability stain solution was prepared using calcein-AM and Ethidium Homodimer-1 (EthD-1) (SIGMA®) (1.6 μM and 4 μM, respectively) and kept away from light at 4° C. until needed for an assay. During each assay, this solution was infused at 10 μL/min for 5 min.

Images were acquired using a monochrome DVC camera connected to an Olympus BX-2 microscope (Center Valley, Pa.) with a 4× objective at a single focal plane at the top of the microcarriers. Monochrome images were obtained for EthD-1 and calcein-AM fluorophores separately using appropriate filter cubes (CHROMA TECHNOLOGY CORK)) at constant exposure rates across all assays and merged into red and green channels of an RGB image. Intensities for both FITC and TxRed channels were measured separately, using a custom written ImageJ macro that extracts the average of the maxima of a series of line profile scans down each microcarrier.

Percent viability was determined for both the BNC platform and a comparative 96-well plate platform using a similar metric by normalizing the fluorescence intensity recorded at the emission wavelength of 645 nm (Ethd-1) relative to positive and negative controls as seen below where F(645)ₛₐₘ is the EthD-1 intensity of the sample at a known doxorubicin concentration and F(645)ₚₒₛ and F(645)_{neg} are the EthD-1 intensities for the positive and negative control, respectively.

$$\% Viability = 1 - ( \frac{{F ( 645 )}_{sam} - {F ( 645 )}_{Pos}}{{F ( 645 )}_{neg} - {F ( 645 )}_{Pos}} \times 100 \% )$$

Negative and positive controls were included as columns in each plate for the 96-well plate assays. Four negative control microcarriers were included in each assay on the BNC platform and one positive control assay served as the positive control for determining percent viability. Dose curves were plotted using a 5-parameter curve fitting algorithm in MATLAB (MATHWORKS®).

An equivalent viability assay was performed using standard 96-well plates for comparative purposes. Cells were seeded on 3, 96-well plates (CORNING®) at a final seeding density of about 10,000 cells per well. Plates were incubated at 37° C., 5% CO₂ for 72 hours before media was exchanged for doxorubicin-containing media and incubated for an additional 24 hours. Negative control columns on each plate were prepared by replacing media with 70% methanol and incubating for 30 minutes at 37° C. Before imaging, all media was removed and cells were washed with warm PBS and replaced with a calcein-AM/EthD-1 solution with the same concentration as in the pBNC assay. Plates were incubated for 20 minutes at room temperature before imaged on a spectrofluorometer (Fluorolog, HORIBA SCIENTIFIC™). A single point scan was performed on each well at excitation/emission wavelengths (nm) of 485/530 for calcein-AM and 530/645 for EthD-1.

Microcarriers used in the nuclear analysis experiments were cultured, harvested, and incubated in different doxorubicin concentrations as described previously in the population live/dead assay. After the 24 hour exposure to doxorubicin, the microcarriers were rinsed with warm 0.1% PBSA and fixed in warmed 0.1% methanol-free formaldehyde in PBS buffer for 1 hour at room temperature. After fixation, the formalin solution was removed and replaced with 0.1% PBSA. The fixed microcarriers were kept at 4° C. until assayed.

Each assay was performed with 12 microcarriers of the same doxorubicin concentration loaded into the wells of a 3×4 micro-container array. The flow cell was assembled as described previously with the micro-container array sandwiched between two fluidic reservoirs.

A nuclear stain was prepared containing 327 μmol/L 4′,6-diamidino-2-phenylindole (DAPI; MOLECULAR PROBES®) in 0.1% PBSA with 0.1% Tween20. The DAPI solution was introduced to the flow cell inlet at a flow rate of 30 μL/min for 2.5 min followed by a wash step of 0.1% PBSA at a flow rate of 30 μL/min for 2.5 min. Images were acquired using a 10× (0.3 NA) objective on an automated Olympus BX-61 modified epifluorescent microscope with motorized stage and 12-bit monochrome CCD camera (Hamamatsu) controlled via HCImage software (Hamamatsu).

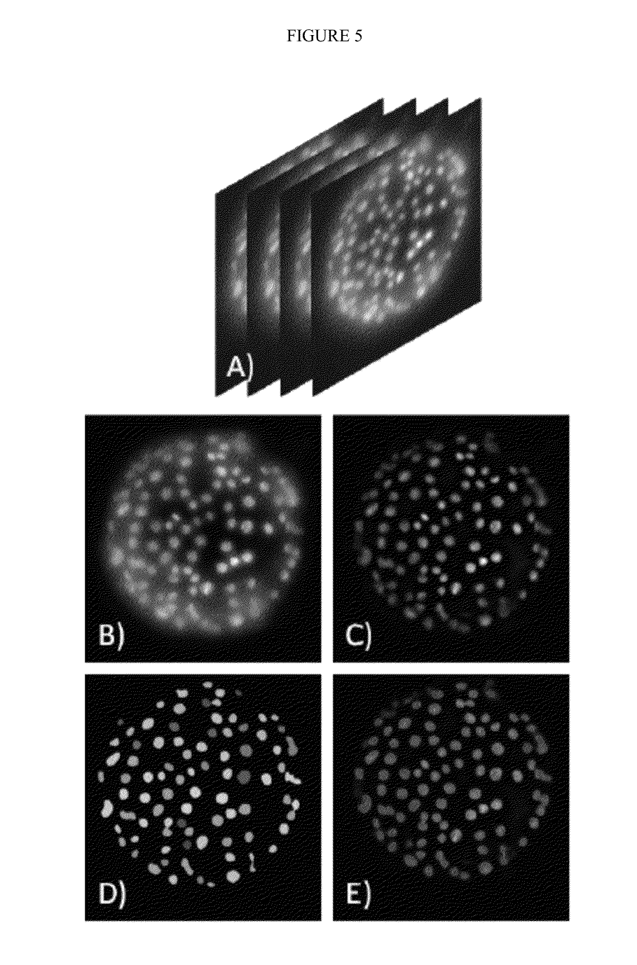

A visual overview of the image processing steps is seen in FIG. 5. Multiple z-focal planes were recorded as TIF-stacks for each microcarrier in a single assay. Each z-stack was comprised of 11 images that spanned a height of 20 μm in increments of 2 μm, where the zero-Z value was defined at the average top focal plane of the microcarrier array. A custom-written macro automated the image acquisition scan of the entire microcarrier array after the x,y,z coordinates were appropriately calibrated. Images were recorded at the same exposure rate for all assays.

A blind deconvolution algorithm was performed on each z-stack of images to improve image contrast. A theoretical point spread function (PSF) was created using the Born and Wolf algorithm under a PSF Generator plugin for ImageJ (BIOMEDICAL IMAGING GROUP, EPFL, Switzerland). The Richardson-Lucy deconvolution algorithm with 10 iterations was performed on each z-stack of images with a Deconvolution Lab plugin for ImageJ (BIOMEDICAL IMAGING GROUP™, EPFL, Switzerland) using the generated PSF as the input PSF.

These image processing steps were performed in a batch process using a FFTW library to increase processing speed. Single maximum intensity projections (MIPs) for each deconvoluted z-stack were obtained and additionally processed with an FFT bandpass filter and background subtraction. Automated image analysis was performed using the open-source software, Cell Profiler, with custom-written macros for contouring cell and nuclear outlines and quantifying fluorescence intensity and various morphological parameters.

An equivalent nuclear assay was performed in monolayer with cells seeded at a density of 5E5 cells per well. Cells were statically cultured for 48 hours followed by a 24 hour exposure to doxorubicin. After fixation, an equivalent DAPI solution was added to each well 5 minutes before imaging to ensure consistent incubation times. Three sets of 12 random images per concentration were recorded using a custom written macro for the microscope stage. Automated image analysis was performed as described above for the microcarrier assay.

Results and Discussion

In our proof of concept studies, we evaluated the use of microcarriers as a potential model for a pseudo-3D in vitro toxicity testing in a programmable-bio-nano-chip platform. Though microcarriers are traditionally used in stirred bioreactor culture, they can also provide higher surface area to volume ratios in static culture. Microcarriers used in the studies presented here were cultured in static environments due to the lower volume requirements for small-scale culture and reduced waste.

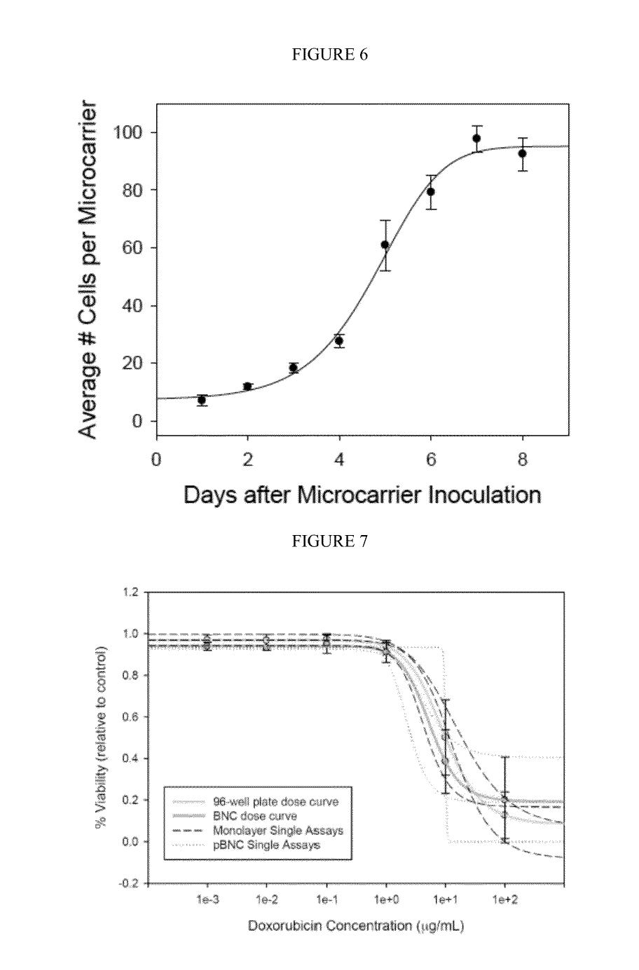

Using seeding densities of 6E4-2.5E5 cells/mL with 5 mg/mL hydrated Cytodex I microcarriers, we were able to achieve confluence after about 5-6 days (FIG. 6). Longer cultures were maintained until about 12-14 days, until cells began to aggregate and grow in multiple layers. We chose our ideal harvest period of between 4 and 6 days due to the well-maintained confluent monolayer present on most microcarriers. Growth dynamics and culture environment was not addressed in this study and will likely be a focus in the future for subsequent assays involving microcarriers.

One of the major limitations of 3D tumor models is image acquisition, as most microscopy techniques that function well for thin, transparent cultures or slices do not function well with the often thick, highly-scattering nature of 3D cultures. By using non-porous, transparent microcarriers, we have proposed an intermediate solution in the transition from 2D culture to truly 3D culture, as these microcarriers offer a “pseudo-3D” environment suitable for image acquisition and analysis. Thus, the techniques described herein could be used for e.g., tissue analysis or levitated cells using magnetic cells and a strong magnetic field.

After addressing the imaging difficulties, the next issue was being able to translate relevant measurements back to well-understood phenomena and cellular models in 2D. By analyzing image stacks recorded for microcarriers with DAPI nuclear staining, we were able to directly compare our results to equivalent assays in 2D. Image processing for each microcarrier included a blind deconvolution process over the entire z-stack, acquiring a maximum intensity projection (MIP) of the deconvoluted z-stack, and image filtering of the resultant MIP image with a bandpass filter and background subtraction (FIG. 5).

One inherent difficulty of imaging isolated microcarriers in a microfluidic environment, is the large variance of hydrated diameters for commercially available Cytodex I microcarriers, translating to a large variance of focal planes for an array of microcarriers. Accordingly, by collecting the same number of images for each microcarrier at equal z-displacements, and performing an iterative deconvolution process, we have reduced the effects of out of focus light blurring in our widefield fluorescence system. To improve imaging variability in the future, these assays should include either a pre-filtering process to isolate and use microcarriers of roughly the same diameter, or they should incorporate an image capture algorithm that acquires a z-stack of images for each microcarrier starting at its own identified focal plane based on its size. Both of these options are currently being pursued.

Even though drug toxicology research is progressing towards the single cell and sub-cellular level, certain parameters describing the effects of a drug on cell populations must be measured from aggregate data. We used a dual-staining method for assessing cellular viability in a 96-well plate format with monolayer cells and the pBNC format with microcarriers. Standard viability kit reagents were used to assess cell viability. Calcein-AM is a non-fluorescent, hydrophobic compound that can permeate live cells and is converted to a highly fluorescent, hydrophilic compound after cleavage by intracellular esterases in viable cells. Ethidium Homodimer-1 (EthD-1) is a cell impermeable, high affinity stain that is weakly fluorescent until bound to either RNA or DNA, and is used as a measure of membrane integrity.

Microcarriers in the pBNC device essentially mimic individual wells of a conventional microtiter plate and could represent unique cell populations, culture conditions, or treatments. By performing the same 24 hour doxorubicin exposure and viability assay, we were able to obtain a representative 5 parameter logistic regression dose-response curve for both platforms. We based our calculation for percent viability off of the fluorescence activity of EthD-1 due to its more reliable signal compared to calcein-AM which can vary from cell to cell by differences in cellular esterase activity and its susceptibility to activation in media and buffer solutions. For an individual cell analysis, this would not be problematic, but for aggregate data these variations cannot be extracted, and can lead to erroneous results.

FIG. 7 shows comparative dose-response curves for MDA-MB-468 cells to doxorubicin in both a monolayer format using a conventional fluorescence plate reader, and a microcarrier format using our pBNC device to perform live/dead assays. Percent viability was determined by normalizing the EthD-1 intensity relative to positive and negative controls. Single dashed or dotted lines represent dose curves computed for individual assays, while the solid, colored lines represent the composite averages across 3 separate assays. Error bars indicate standard deviations across assays repeated in triplicate. Computed IC₅₀ values for the composite curves are 4 μL/mL for the BNC assays and 10 μL/mL for the 96-well plate assays. A comparison of computed dose curves in FIG. 7 showed similar curves for both platforms with a measured IC₅₀ value within an order of magnitude. Therefore, the ability of the pBNC card to assess cell viability has been well demonstrated.

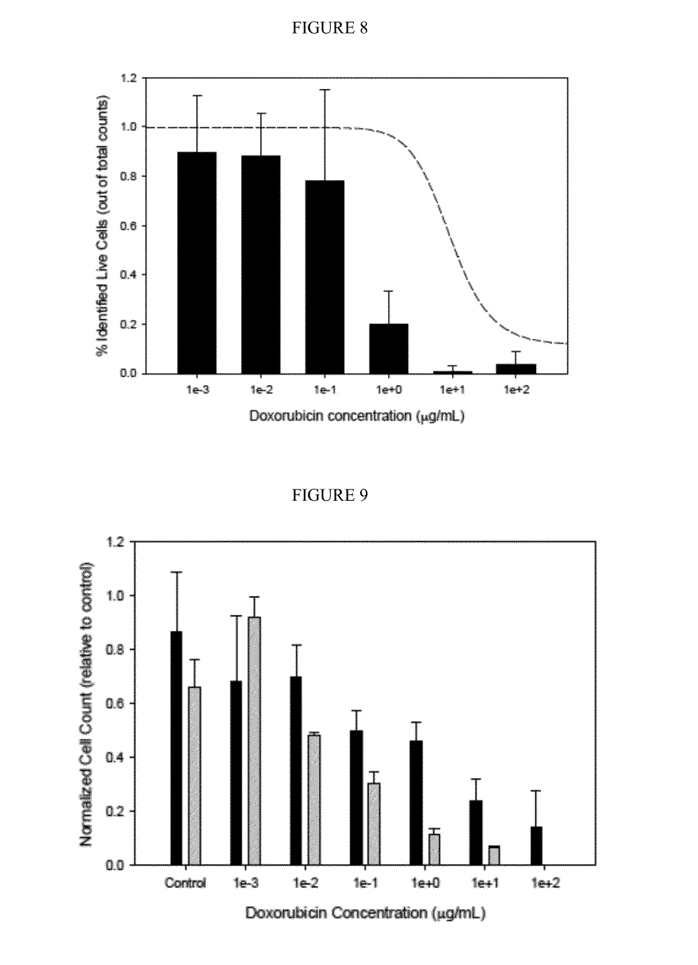

FIG. 8 demonstrates how the ability to isolate single cells in a commonly used population assay can yield additional metrics for computing cell viability. In this example, images recorded from a live/dead assay at 5× magnification were analyzed in Cell Profiler where single cells appearing green (false-colored calcein-AM fluorescence) or red (false-colored EthD-1 fluorescence) were outlined for each microcarrier in an array on the pBNC platform. Percent viability was then calculated as an overall percent of identified green cells over the total number of cells for each microcarrier. Error bars for this measurement represents the intra-assay variability between microcarriers for different doxorubicin concentrations. These error bars appear much larger than for averaged intensity measurements, seen in FIG. 7, because percent viability determined from cell counting is essentially a binary measure and fails to account for cells with calcein-AM or EthD-1 activity below the threshold established for outlining cells. In this case, the population data based on average fluorescence would be a more useful measure of percent viability, but individual cell counting could provide additional information at the single-cell level.

FIG. 9 shows another way to measure cell counts wherein total cell counts for microcarrier and monolayer assays are based on identified nuclear staining (gray=monolayer assay, black=microcarrier assay). Cell counts were normalized by dividing by average control counts for each respective platform and scaling between 0 and 1. Error bars represent one standard deviation in each data set repeated in triplicate.

Cell counted by the two different methods (percent viability by calcein-AM/EthD-1 stain, and total cells by DAPI-stained nuclei in a fixed cell assay) both yielded comparable results, each showing a decrease in total cell population for increasing doxorubicin concentrations. Like standard tissue culture plates, adherent cells on microcarriers exposed to high levels of toxic compounds dissociate from the culture surface.

Nuclear morphometry has been implicated as a prognostic variable capable of providing information to predict certain chemotherapy responses. In a nuclear analysis comparison between the pBNC platform and monolayer culture involving fixed cells and DAPI-staining, several trends related to nuclear morphology, nuclear intensity, and overall cell counts were found to be comparable. FIG. 9 shows the comparison between identified nuclear counts for microcarriers and a comparable assay for monolayer cells. At doxorubicin concentrations of 100 μg/mL, no monolayer cells remained attached after 24 hour exposure, though some cells remained attached to Cytodex I microcarriers.

Reasons for this may include differences in material composition that result in higher binding affinity for MDA-MB 468 cells to Cytodex I microcarriers than standard tissue culture-treated polystyrene, though this has yet to be studied. Another reason for this may be due to the lower shearing associated with the wash step with microcarriers, where a protective layer of microcarriers forms around remaining microcarriers settled in solution, compared to the direct washing in monolayer culture.

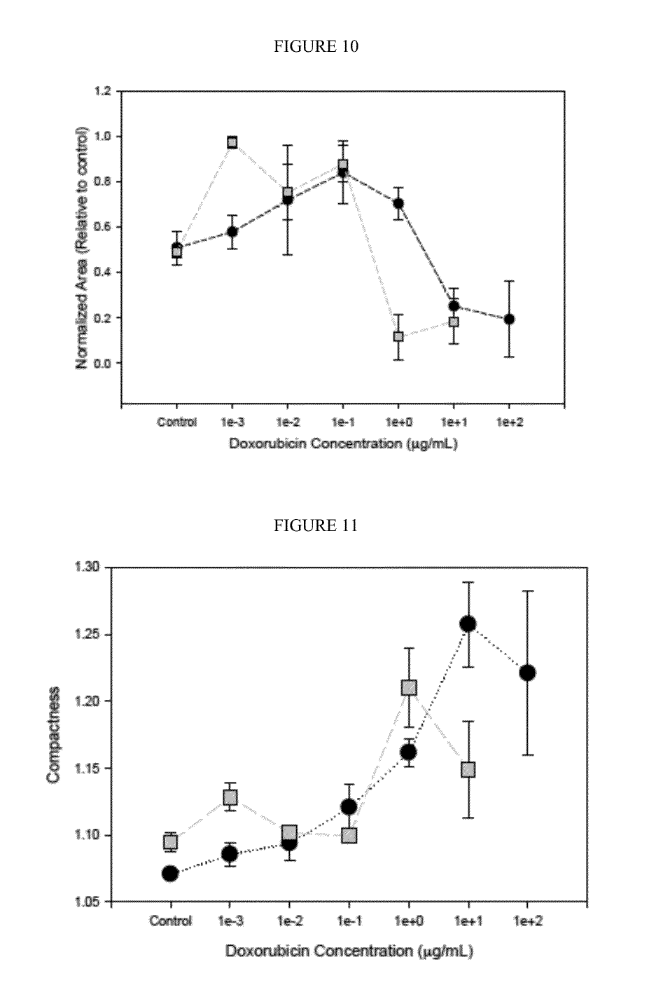

Using an automated cell profiling and measurement algorithm in Cell Profiler^{[1, 2]}, we were able to obtain morphological and intensity measurements for DAPI-stained nuclei on microcarriers and monolayer cells at various doxorubicin concentrations. When comparing nuclear area between microcarriers and monolayer cells, we observed that both followed a similar decreasing trend with an increasing concentration of doxorubicin, as seen in FIG. 10.

In FIG. 10 changes in nuclear area with increasing doxorubicin concentration (Gray with squares=monolayer assay, Black with circles=microcarrier assay) are shown. Nuclear area was computed in Cell Profiler from identified nuclear ROI's, normalized by dividing by average control areas, and scaled between 0 and 1. There is no data for monolayer cells at 100 μg/mL since no adherent cells were identified for this condition.

The average nuclear area increases for low concentrations of doxorubicin and decreases for higher concentrations, giving the impression of an overall decrease in nuclear area across a 10.000-fold increase in doxorubicin concentration. This biphasic trend is consistent with accepted theory that low compound doses can induce cell-cycle arrest while higher compound doses indicate apoptotic death. Cell-cycle arrest leads to an increase in nuclear area via mitotic inhibition while apoptotic death leads to a decrease in nuclear area due to nuclear fragmentation. Further confirmation of these events could be performed by 5-bromo-2-deoxyuridine (BrdU) labeling to detect cell-cycle arrest through and increase in cyclin B1-positive cells and a caspase-3 activation assay to detect apoptosis initiation. This trend was observed for both microcarrier and monolayer platforms.

Nuclear compactness, another measure of nuclear morphology, was computed from identified nuclear ROI's as a measure of the variance in the radial distance of an object's pixels from its centroid divided by its area (FIG. 11). Smaller values for compactness indicate a lower variance in the spread of pixels from the object's centroid while larger values indicate a less uniform morphology.

We observed an increase in the average uncorrected compactness for both the microcarrier and monolayer format with increasing doxorubicin concentration. This observation is possibly due to the increase in cytotoxicity which can disrupt the normal, compact nuclear morphology and result in nuclear fragmentation and other variations in nuclear spread. Nuclear compactness has been indicated as a prognostic marker when combined with other nuclear features, but has not been extensively studied on its own.

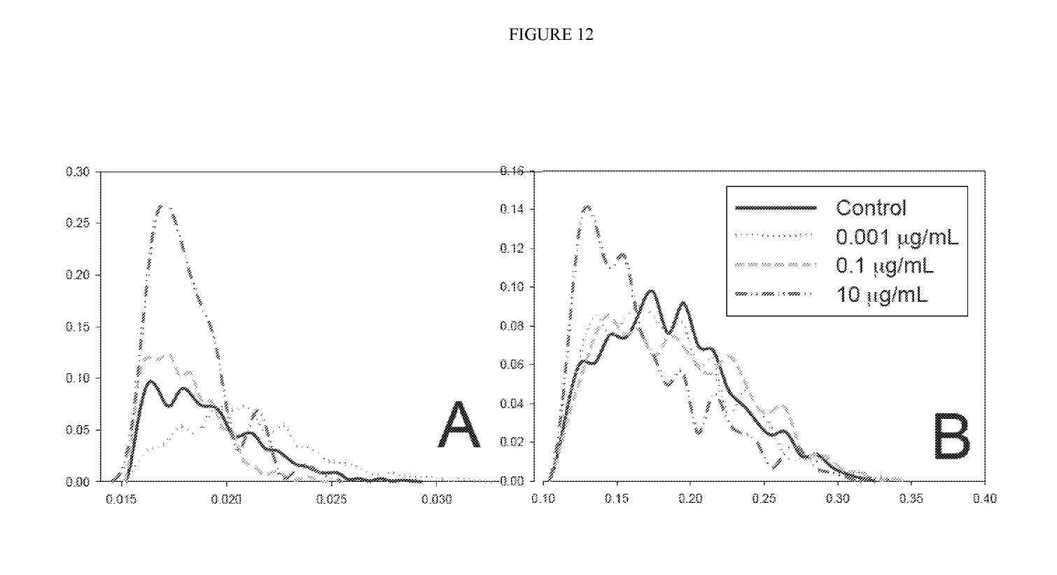

In addition to morphometric comparisons, DAPI intensity was also used to compare the response of both platforms to a range of doxorubicin concentrations (FIG. 12). Average DAPI intensity was calculated by dividing the integrated intensity of the identified nuclear ROI's by their corresponding area. Normalized histograms depicting the distribution of the average DAPI intensity for each platform were plotted for a range of doxorubicin concentrations.

Overall, both platforms display a shift in the distribution of DAPI intensities towards lower intensities for high doxorubicin concentrations (10 μg/mL). Mean DAPI intensity has been used as a direct measure of DNA content in cells for decades, and is commonly used to differentiate between different cell-cycle phases when analyzing the distribution of nuclear intensities in a sample. Since doxorubicin and DAPI are both known DNA intercalating agents, we believe one reason for the perceived decrease in DAPI staining intensity is competitive DNA intercalation. At higher concentrations of doxorubicin, there is a higher degree of DNA intercalation and therefore fewer binding sites for DAPI to bind, reducing the amount of fluorescently active DAPI in solution. In order to assess the effect of cell-cycle inhibition on the change in nuclear intensity distribution, future assays could incorporate a known mitotic inhibitor drug, such as colchicine, or additional cell-cycle phase-specific reagents, such as BrdU.

Herein, we have presented an introductory comparison of several predictive toxicology assay endpoints between pseudo-3D microcarrier cultured cells in a programmable-bio-nano-chip platform and traditional monolayer culture. While equivalent tumor models take on many forms over a diverse range of materials and culture methods, they all suffer from difficulties in imaging, making comparative measurements to standard monolayer culture, and sequestering 3D scaffolds for multiplexed analysis. We have addressed each of these issues by developing a microfluidic platform that enables automated image capture and analysis of individually addressable cell-cultured microcarrier units. Even though non-porous microcarriers are traditionally used for large scale industrial production of biologics, they present a simple transition into high content analysis of 3D cultured toxicology assays. Future work will incorporate further microfluidic operations into this platform, such as automated microcarrier harvesting and transport to assay zones and on-chip dosing of multiple compounds in order to increase the automation of individualized toxicology assays.

Monolayer Cultures

The devices described herein can also be used for traditional 2D time course assays. Using patient-derived GBM-4687 glioblastoma cells cultured in DMEM with 10% FBS incubated for 24 hours at 37° C. and 5% CO₂ at a density of 1E5 cells/mL, we showed that both culture platforms display equivalent cell attachment characteristics and distribution (data not shown). Composite images show similar cell arrangement and morphologies and support the ability to perform fixed cell analysis on the microfluidic culture device.

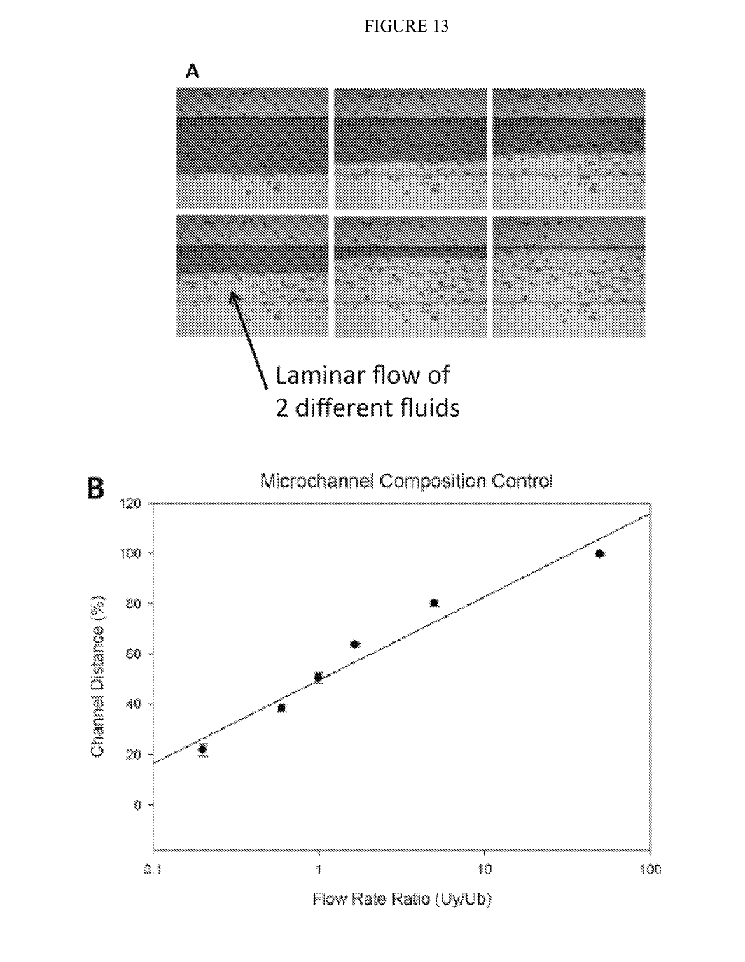

We also showed controlled sequestered reagent delivery to a single well via laminar flow in FIG. 13. In FIG. 13A, two different inks under separate pumps were fed into an elongated chamber of the card shown in FIG. 3, where the well was 30 mm long 3 mm wide, and 200 μm deep. The pumps were programmed to initiate flow with predominantly dark in (first panel) to predominantly light ink (last panel), passing through a point indicated by the arrow where the two flow rates were equal. These images demonstrated the ability to control the position of two (or more) separate fluids in order to sequester a reagent to a particular channel width.

FIG. 13B is a linear regression of channel distance as a function of the flow rate ratio between the two independent fluids (attached to independent pumping sources). Data was collected in triplicate and error bars represent one standard deviation above and below the mean for each flow rate ratio.

Thus, we were able to show that we can test multiple different reagents or drugs across cells in the same growth environment. This can be useful for running different controls simultaneously across cells grown under the same microenvironment conditions. Such laminar flow could find use in methods such as hormone testing, drug screening, or cell mobility assays.

We also demonstrated that our monolayer microfluidic chip had utility in a wound-scratch/migration assay through trypsinization. A converging channel design (e.g., FIG. 3) for the monolayer device where 3 (or 2) channels converge into a single microfluidic assay chamber allows for efficient and rapid set-up for wound-scratch or migration assays where cells are cultured until an adherent monolayer forms, and then a particular region is treated until the cells in that region detach and wash away. Cell regrowth is then monitored as a surrogate for wound healing.

In our experiments (not shown), three distinct zones are illustrated after only 5 minutes of treatment with a single streamline of trypsin flowing along a region of buffer in parallel (across still-adherent cells). Because the simultaneously perfused fluid streams observe laminar flow, a centered narrow fluid stream of trypsin is “focused” between two outer sheath fluids and is passed over the adherent cells in the main channel. The width of this channel can be easily characterized and measured based on the relationship shown in FIG. 13B. After the trypsin-treated cells have detached, the channel is perfused with standard media and time-course imaging is performed to observe the cells returning over the void area. While these “scratch-wound” assays are typically performed in standard petri dishes with a manual scratch process where a pipette tip is used to create a void area in an adherent layer of cells, the quantification of such assays is difficult to perform due to the high variability in creating the void region. Therefore, the pNBC card can be used for 2D assays, such as a wound healing assay.

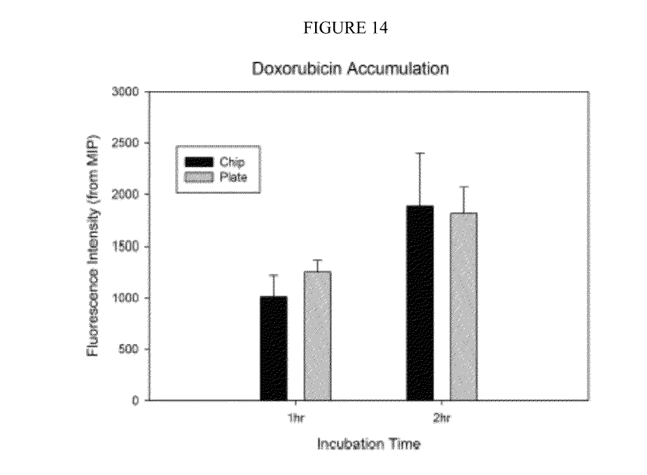

FIG. 14 shows the comparison between chip and static doxorubicin uptake in a bar diagram. The results are highly comparable, demonstrating the usefulness of the platform to toxicity assays.

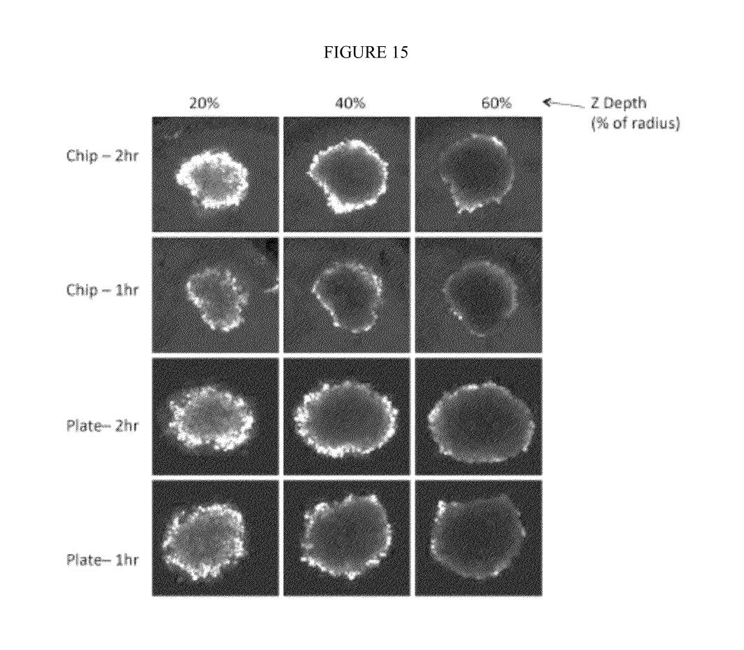

FIG. 15 shows the difference between chip and plate doxorubicin uptake with different Z depth (% radius). This assay shows the similarity in drug uptake as a function of position on the spheroid microcarrier between our dosing platform and standard culture platforms. The different z-slices demonstrate how doxorubicin is only able to penetrate through a few outer layers of cells during 2 hours incubation, which is observed for both platforms. This is important because it demonstrates that with our active fluid-pumping approach, the biological response is not changed by forcing extra drug inside the spheroid.

Each of the following reference is incorporated by reference herein in its entirely.
1. Carpenter, A. E., et al., CellProfiler: image analysis software for identifying and quantifying cell phenotypes. Genome Biology, 2006. 7(10).
2. Lamprecht, M. R., D. M. Sabatini, and A. E. Carpenter, CellProfiler™ free, versatile software for automated biological image analysis. Biotechniques, 2007. 42(1): p. 71-75.
61/412,994 filed on Nov. 12, 2010; 61/413,107 filed on Nov. 12, 2010; 61/498,761 filed on Jun. 20, 2011; 61/484,492 filed on May 10, 2011; 61/485,189 filed on May 12, 2011; 61/558,165 filed on Nov. 10, 2011; 61/638,264 filed on Apr. 25, 2012; 61/815,902 filed on Apr. 23, 2013; 61/816,083 filed on Apr. 25, 2013; 61/841,090 filed on Jun. 28, 2013; Ser. No. 14/114,925 filed on Mar. 21, 2013; Ser. No. 14/258,770 filed on Apr. 22, 2014; Ser. No. 14/261,670 filed on Apr. 25, 2014
US20120322682, WO2012154306, WO2012065117, WO2012065025, WO2012021714, WO2007134189, and WO2012065025
U.S. Ser. No. 14/258,770, filed Apr. 22, 2014, based on 61/815,305, filed Apr. 24, 2013.

1. A disposable labcard comprising:
a) a multilayered top compartment and a multilayered bottom compartment held in leakproof juxtaposition and comprising a plurality of fluidic pathways;
b) wherein said top compartment comprises inlets, vents and outlets,
c) said bottom compartment comprises a plurality of wells, each well having an open top and a flat transparent bottom; and,
d) wherein each fluidic pathway includes at least one inlet, at least one vent, at least one well and at least one outlet.

2. The disposable labcard of claim 1, wherein said fluidic pathway comprised a plurality of inlets each connected to a vent, and said plurality of inlets merge to connect to a single well.

3. The disposable labcard of claim 1, wherein each said fluidic pathway comprises at least two wells in series allowing each assay to be performed in duplicate.

4. The disposable labcard of claim 2, wherein said fluidic pathway comprises at least two wells in series allowing each assay to be performed in duplicate.

5. The disposable labcard of claim 1, wherein each said fluidic pathway comprises at least three wells in series allowing each assay to be performed in triplicate.

6. The disposable labcard of claim 2, wherein each said fluid pathway comprises at least three wells in series allowing each assay to be performed in triplicate.

7. The disposable labcard of claim 1, wherein said multilayered top compartment and a multilayered bottom compartment each comprise polymeric layers.

8. The disposable labcard of claim 7, wherein said polymeric layers are selected from a group consisting of DSA (double sided adhesives), PET (polyethylene terephthalate), PMMA (poly(methyl methacrylate)), cyclic olefin co-polymer (COC) and PVDF (polyvinylidene difluoride).

9. The disposable labcard of claim 7, wherein said polymeric layers are alternating layers of DSA and PET.

10. The disposable labcard of claim 7, wherein each vent comprises a gas pathway to an upper surface of said disposable card above an inlet pathway, said gas pathway having a porous membrane thereacross allowing gas passage therethrough.

11. The disposable labcard of claim 7, wherein each well comprises a plurality of microcarrier beads for growing cells on surfaces thereof.

12. A microassay platform comprising:
a) the mutilayered disposable labcard of claim 1;
b) a card holder having a pocket for said disposable labcard and having a footprint of a standard microtiter plate;
c) a reusable fluidic manifold located above said disposable labcard and in fluid connection with said disposable card and having leakproof fluidic connections with one or more outside fluid sources; and,
d) holding means for holding said labcard holder, said disposable card, and said reusable fluidic manifold together.

13. A platform for performing assays, said platform comprising:
a) a reusable fluidic manifold;
b) a disposable card having a plurality of fluidic pathways;
i) wherein said disposable labcard comprises a multilayered top compartment and a multilayered bottom compartment;
ii) wherein said top compartment comprises inlets, vents and outlets;
iii) wherein said bottom compartment comprises wells, each well having a flat transparent bottom;
iv) wherein each fluidic pathway includes at least one inlet and vent connected to at least one well connected to at least one outlet;
v) wherein said fluidic manifold provides a plurality of connectors, each allowing fluid flow between an external fluid source and one of said plurality of fluidic pathways;
c) a card holder having a footprint of a standard microtiter plate and a pocket therein side to receive said disposable labcard; and,
d) a means for holding said reusable fluidic manifold, said disposable labcard and said card holder together in leak proof juxtaposition.

14. The platform of claim 13, wherein each fluidic pathway includes a plurality of inlets connected to said at least one well.

15. The platform of claim 13, wherein each fluidic pathway includes an inlet connected to a plurality of wells in series.

16. The platform of claim 13, wherein each fluidic pathway includes a plurality of inlets connected to a first well, and said first well connected at least one second well.

17. A method of performing assays on cell cultures, comprising:
a) providing a platform of claim 12,
b) inoculating said wells with a population of cells;
c) continuously perfusing a cell medium through said fluidic pathways; and,
d) measuring one or more parameters of said cells over time.

18. The method of claim 17, wherein a test agent is added to said cell medium and measuring said one or more parameters of said cells again.

19. The method of claim 17, wherein said more or more parameters is selected from cell number, viable cell number, nuclear size, dead cell number, cell area, and nuclear-to-cytoplasm area.
